(19) United States
(12) Patent Application Publication
(10) Pub. No.: US 2022/0367043 A1
(43) Pub. Date: Nov. 17, 2022

(54) ENHANCED ELECTRONIC WHITEBOARDS FOR CLINICAL ENVIRONMENTS

(71) Applicant: Hill-Rom Services, Inc., Batesville, IN (US)

(72) Inventors: Susan A. Kayser, Batesville, IN (US); Lori A. Zapfe, Milroy, IN (US); Kelli F. Rempel, Chapel Hill, NC (US); Michael S. Hood, Batesville, IN (US); Georg Köllner, Bad Tabarz (DE); Sinan Batman, Cary, NC (US); Jennifer Marie Rizzo, Guilford, IN (US); Mary L Pfeffer, Mount Pleasant, SC (US); Jie Zhou, Batesville, IN (US); Nuno M Azeredo, Chicago, IL (US); Mary Markham-Feagins, Batesville, IN (US)

(21) Appl. No.: 17/743,440

(22) Filed: May 12, 2022

Related U.S. Application Data
(60) Provisional application No. 63/189,042, filed on May 14, 2021.

(51) Int. Cl. G16H 40/63; G16H 10/60

(52) U.S. Cl. CPC G16H 40/63; G16H 10/60

(57) ABSTRACT

An example method includes outputting, by an output device, a first Graphical User Interface (GUI) associated with a patient. A font size of the first GUI is adjusted based on a position of the patient. Based on determining that a position of a care provider is within the threshold distance of the output device or within the room associated with the patient, outputting, by the output device, the second GUI being different than the first GUI.

### CROSS-REFERENCE TO RELATED APPLICATION

This application claims the priority of U.S. Provisional Application No. 63/189,042, which was filed on May 14, 2021 and is incorporated by reference herein in its entirety.

### TECHNICAL FIELD

This application relates generally to electronic whiteboards that can be used to provide and/or receive clinical information. In some cases, the electronic whiteboards can automatically interface with Electronic Medical Records (EMRs).

### BACKGROUND

In clinical, in-patient environments, patient rooms are typically outfitted with manual whiteboards. These whiteboards may be dry-erase boards that care providers may use to update the patients about their ongoing care. In some cases, the whiteboards indicate information such as identities of care providers responsible for the patient's care (e.g., a charge nurse, a certified nurse assistant (CNA), a physician, etc.), contact information for the care providers, simple plans of care, and simple patient goals.

However, in real-world environments, manual whiteboards often display inaccurate information. For example, care providers may forget to update manual whiteboards after shift changes. Since updating manual whiteboards is another burdensome task to already overloaded schedules of care providers, some care providers may refrain from filling out or updating manual whiteboards in lieu of higher priority tasks, such as patient care and electronic medical record (EMR) documentation. As a result, patients and patient family members may be unable to identify patient statuses or care providers who should be contacted in view of patient status changes.

Furthermore, manual whiteboards can be a contamination risk for clinical environments. In addition, scents associated with dry-erase boards can be problematic for individuals with pulmonary problems, such as asthma and chronic obstructive pulmonary disease (COPD).

### SUMMARY

Various implementations of the present disclosure relate to electronic whiteboards configured to output patient-related information to patients, patient family members, and care providers. In particular examples, an electronic whiteboard is physically mounted in a room of a patient. The electronic whiteboard may present information that is relevant to the patient, family members of the patient, and other non-clinical individuals caring for the patient. In some cases, the electronic whiteboard may identify a care provider in the vicinity of the electronic whiteboard. Based on identifying the care provider, the electronic whiteboard may display information that is relevant to the care provider, which may be different than the information that is relevant to the patient. Thus, the electronic whiteboard can be adaptively used by patients and care providers to identify information relevant to ongoing care of the patient.

In various cases, the electronic whiteboard may interface directly with the electronic medical record (EMR) of the patient. The electronic whiteboard may automatically update information output to the patient or care provider based on data from the EMR of the patient. In some cases, the electronic whiteboard may present accurate information based on real-time updates to the EMR. Thus, the electronic whiteboard is less likely to present out-of-date information than manual whiteboards. Furthermore, the electronic whiteboard may receive input signals directly from the patient and/or care provider and may automatically update the EMR of the patient based on the input signals. Accordingly, the electronic whiteboard may provide a convenient portal through which the EMR of the patient can be updated.

In some examples, the EMR and/or the information output by the electronic whiteboard can be updated in a hands-free fashion. For instance, the electronic whiteboard may interface with or include a microphone configured to receive a voice input or a camera configured to detect a hand gesture from the patient and/or care provider. The electronic whiteboard may update the EMR and/or the information output by the electronic whiteboard based on the voice input and/or hand gesture. Thus, the electronic whiteboard may receive commands from the patient and/or care provider without the patient and/or care provider touching the electronic whiteboard, thereby reducing a risk of fomite-based transmission within the clinical environment. Furthermore, the care provider may provide verbal commands or updates to the electronic whiteboard while the care provider's hands are otherwise occupied, such as during emergency situations in which the care provider is performing cardiopulmonary resuscitation (CPR) on the patient.

The electronic whiteboard, in some cases, may adapt according to the position and/or languages spoken by the patient and care provider. In some examples, the electronic whiteboard presents text and/or graphics, and may adjust the size of the text and/or graphics based on the proximity of the patient or care provider to the electronic whiteboard. In some cases, the electronic whiteboard detects a language spoken by the patient or care provider and automatically presents text or audibly outputs words in the detected language. The electronic whiteboard may, in some examples, translate between different languages spoken by the patient and care provider.

According to some implementations, the electronic whiteboard may provide pertinent information about the patient on a single user interface when the patient is in need of emergency care. For example, when the patient is experiencing cardiopulmonary arrest, the patient may be in immediate need of resuscitative care. In these circumstances, the electronic whiteboard may present highly relevant information to the resuscitation that the care provider may not have otherwise had enough time to view or review prior to beginning the resuscitation. For instance, the electronic whiteboard may indicate whether the patient has a do-not-resuscitate (DNR) order, pertinent allergies to medicines that may be administered during resuscitation, other care providers who must be contacted immediately in emergencies associated with the patient, and so on.

Various implementations described herein address specific problems in the technical field of medicine. Various electronic whiteboards described herein provide accurate, up-to-date information for patients and care providers, which is an improvement over manual whiteboards that often display inaccurate and/or outdated information. Furthermore, electronic whiteboards can directly update the EMRs of patients, which can reduce the time that care providers spend charting. In particular cases, electronic whiteboards may be hands-free, which may reduce the risk of contamination in a clinical environment. Accordingly, various implementations have practical applications to clinical care environments.

### DESCRIPTION OF THE FIGURES

The following figures, which form a part of this disclosure, are illustrative of described technology and are not meant to limit the scope of the claims in any manner.

FIGS. 1A and 1B illustrate an example clinical environment at different times.

FIGS. 2A and 2B illustrate examples of different graphical user interfaces that may be output by a screen of an electronic whiteboard.

FIG. 3 illustrates an example of an emergency GUI that can be output by the electronic whiteboard.

FIG. 4 illustrates an example environment in which an augmented reality (AR) device is used to provide patient-relevant information to a care provider.

FIG. 5 illustrates an example process for outputting patient information on an electronic whiteboard.

FIG. 6 illustrates at least one example device configured to enable and/or perform the some or all of the functionality discussed herein.

### DETAILED DESCRIPTION

Various implementations of the present disclosure will be described in detail with reference to the drawings, wherein like reference numerals present like parts and assemblies throughout the several views. Additionally, any examples set forth in this specification are not intended to be limiting and merely set forth some of the many possible implementations.

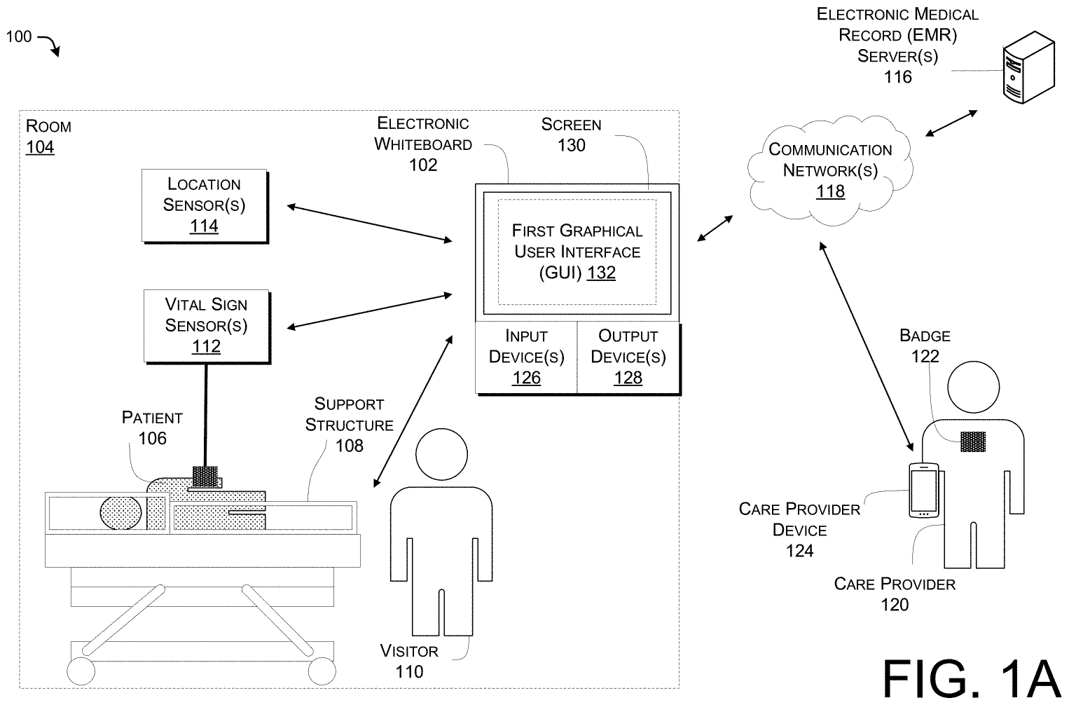

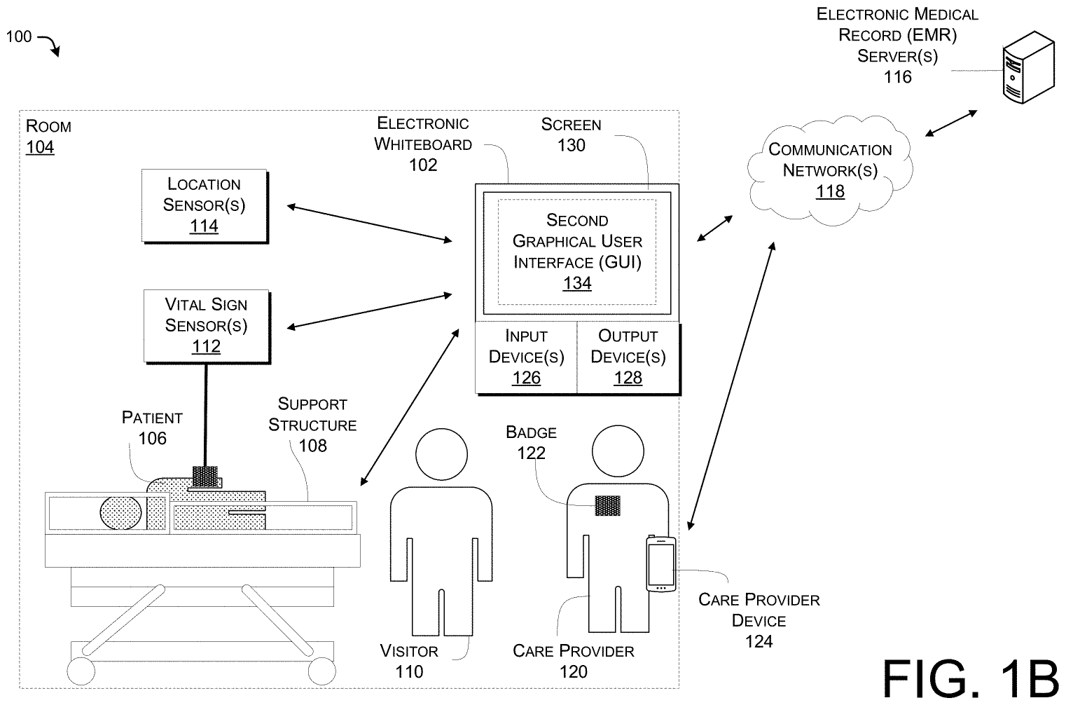

FIGS. 1A and 1B illustrate an example clinical environment 100 at different times. The clinical environment 100, for instance, may be a hospital, a clinic, a care facility, or a combination thereof. In particular, FIG. 1A illustrates the clinical environment 100 at a first time and FIG. 1B illustrates the clinical environment 100 at a second time. The first time may occur before or after the second time, in various cases.

The clinical environment may include an electronic whiteboard 102 located in a room 104 associated with a patient 106. The electronic whiteboard 102 may be implemented by one or more computing devices. As used herein, the term “computing device,” and its equivalents, may refer to a device including at least one processor configured to perform predetermined operations. Examples of computing devices include mobile phones, tablet computers, personal computers, laptops, and smart televisions. As used herein, the term “patient,” and its equivalents, can refer to an individual being monitored and/or cared for within a clinical environment or who has been previously monitored and/or cared for within the clinical environment. In various examples, a patient is a human, but implementations of this disclosure are not so limited. In general, the electronic whiteboard 102 may be configured to output information relevant to the patient 106 or care of the patient 106 in the clinical environment 100.

The room 104 may include an at least partially enclosed space within the clinical environment. In some cases, the room 104 may be shared between the patient 106 and one or more additional patients in the clinical environment 100. In some cases, the room 104 may include a permanent structure, such as a wall or other installation, on which the electronic whiteboard 102 is physically attached. For example, the electronic whiteboard 102 may be physically mounted on a wall in the room 104.

In various cases, the patient 106 may be assigned the room 104 within the clinical environment 100. For example, the patient 106 may reside in the room 104 for an extended period of time, such as for one hour, ten hours, one day, ten days, etc. In various cases, the room 104 may be part of an intensive care unit (ICU) in the clinical environment 100, or some other in-patient area of the clinical environment 100.

The patient 106 may rest on a support structure 108. The support structure 108, for instance, may include a gurney, hospital bed, or some other structure configured to support the patient 106. As used herein, the terms “bed,” “hospital bed,” and their equivalents, can refer to a padded surface configured to support a patient for an extended period of time (e.g., hours, days, weeks, or some other time period). The patient 106 may be laying down on the support structure 108. For example, the patient 106 may be resting on the support structure 108 for at least one hour, at least one day, at least one week, or some other time period. In various examples, the patient 106 and the support structure 108 may be located in the room 104. In some implementations, the support structure 108 includes a mechanical component that can change the angle at which the patient 106 is disposed. In some cases, the support structure 108 includes padding to distribute the weight of the patient 106 on the support structure 108. According to various implementations, the support structure 108 can include vital sign monitors configured to output alarms or otherwise communicate vital signs of the patient 106 to external observers (e.g., care providers, family members, and the like). The support structure 108 may include railings that prevent the patient 106 from sliding off of a resting surface of the support structure 108. The railings may be adjustable, in some cases.

In various examples, the support structure 108 includes one or more sensors. For instance, the support structure 108 may include one or more load cells. The load cell(s) may be configured to detect a pressure on the support structure 108. In various cases, the load cell(s) can include one or more strain gauges, one or more piezoelectric load cells, a capacitive load cell, an optical load cell, any device configured to output a signal indicative of an amount of pressure applied to the device, or a combination thereof. For example, the load cell(s) may detect a pressure (e.g., weight) of the patient 106 on the support structure 108. In some cases, the support structure 108 includes multiple load cells that respectively detect different pressures on the support structure 108 in different positions along the support structure 108. In some instances, the support structure 108 includes four load cells arranged at four corners of a resting surface of the support structure 108, which respectively measure the pressure of the patient 106 on the support structure 108 at the four corners of the support structure 108. The resting surface, for instance, can be a surface in which the patient 106 contacts the support structure 108, such as a top surface of the support structure 108.

The support structure 108 may include one or moisture sensors. The moisture sensor(s) may be configured to measure a moisture on a surface (e.g., the resting surface) of the support structure 108. For example, the moisture sensor(s) can include one or more capacitance sensors, one or more resistance sensors, one or more thermal conduction sensors, or a combination thereof. In some cases, the moisture sensor(s) include one or more fiber sheets configured to propagate moisture to the moisture sensor(s). In some cases, the moisture sensor(s) can detect the presence or absence of moisture (e.g., sweat or other bodily fluids) disposed between the support structure 108 and the patient 106.

In various examples, the support structure 108 can include one or more temperature sensors. The temperature sensor(s) may be configured to detect a temperature of at least one of the patient 106, the support structure 108, or the room 104. In some cases, the temperature sensor(s) includes one or more thermistors, one or more thermocouples, one or more resistance thermometers, one or more Peltier sensors, or a combination thereof.

The support structure 108 may include one or more cameras. The camera(s) may be configured to capture images of the patient 106, the support structure 108, the room 104, or a combination thereof. In various cases, the camera(s) may include radar sensors, infrared cameras, visible light cameras, depth-sensing cameras, or any combination thereof. In some examples, infrared images may indicate, for instance, a temperature profile of the patient 106 and/or the support structure 108. Thus, the camera(s) may be a type of temperature sensor. In addition, the images may indicate a position of the patient 106 and/or the support structure 108, even in low-visible-light conditions. For example, the infrared images may capture a position of the patient 106 during a night environment without ambient lighting in the vicinity of the patient 106 and/or the support structure 108. In some cases, the camera(s) may include one or more infrared video cameras. The camera(s) may include at least one depth-sensing camera configured to generate a volumetric image of the patient 106, the support structure 108, and the ambient environment. According to various implementations, the images and/or videos captured by the camera(s) are indicative of a position and/or a movement of the patient 106 over time.

According to some examples, the support structure 108 can include one or more video cameras. The video camera(s) may be configured to capture videos of the patient 106, the support structure 108, the room 104, an entrance to the room 104, an entrance to a bathroom adjacent to the room 104, or a combination thereof. The videos may include multiple images of the patient 106 and/or the support structure 108. Thus, the videos captured by the video camera(s) may be indicative of a position and/or movement of the patient 106 over time. In some examples, the video camera(s) capture visible light videos, changes in radar signals over time, infrared videos, or any combination thereof.

In some examples, the support structure 108 can include one or more microphones configured to capture audio signals output by the patient 106, the support structure 108, and/or the ambient environment. The audio signals captured by the microphone(s) may be indicative of a position and/or movement of the patient 106 over time. In particular cases, the microphone(s) are integrated within the camera(s) and/or video camera(s).

In some examples, the support structure 108 includes a head rail and a foot rail. The camera(s) and/or video camera(s), for instance, are mounted on the head rail, the foot rail, an extension (e.g., a metal or polymer structure) attached to the head rail or the foot rail, or any combination thereof. In various implementations, the camera(s) and/or video camera(s) are attached to a wall or ceiling of the room containing the support structure 108. In some examples, the camera(s) and/or video camera(s) are attached to a cart or other object that is located in the vicinity of the support structure 108. In some implementations, the camera(s) and/or video camera(s) are integrated with the electronic whiteboard 102.

In various cases, the sensors (e.g., the load cell(s), the moisture sensor(s), the temperature sensor(s), the camera(s), the video camera(s), the microphone, or any combination thereof) of the support structure 108 are configured to monitor one or more parameters of the patient 106 and to generate sensor data associated with the patient 106. In various cases, the sensors convert analog signals (e.g., pressure, moisture, temperature, light, electric signals, sound waves, or any combination thereof) into digital data that is indicative of one or more parameters of the patient 106. As used herein, the terms “parameter,” “patient parameter,” and their equivalents, can refer to a state of an individual and/or the surrounding environment. In this disclosure, a parameter of the patient 106 can refer to a position of the patient 106, a movement of the patient 106 over time (e.g., mobilization of the patient 106 on and off of the support structure 108), a pressure between the patient 106 and an external object (e.g., the support structure 108), a moisture level between the patient 106 and the support structure 108, a temperature of the patient 106, a vital sign of the patient 106, a nutrition level of the patient 106, a medication administered and/or prescribed to the patient 106, a previous state of the patient 106 (e.g., the patient was monitored in an ICU, in dialysis, presented in an emergency department waiting room, etc.), circulation of the patient 106 (e.g., restricted blood flow), a pain level of the patient 106, the presence of implantable or semi-implantable devices (e.g., ports, tubes, catheters, other devices, etc.) in contact with the patient 106, a sound emitted by the patient 106, or any combination thereof. In various examples, the load cell(s), the moisture sensor(s), the temperature sensor(s), the cameras, the video camera(s), the microphone(s), or a combination thereof, generates sensor data indicative of one or more parameters of the patient 106. The support structure 108 may (e.g., periodically) transmit the sensor data to the electronic whiteboard 102.

A visitor 110 may also be located in the room 104 of the patient 106. The visitor 110 may be an individual concerned with the health of the patient 106, but who is not a care provider in the clinical environment 100. For example, the visitor 110 may be a friend, a loved one, or a family member of the patient 106. Although only a single visitor 110 is illustrated in FIGS. 1A and 1B, implementations are not so limited. For example, multiple visitors 110 of the patient 106 may be located in the room 104.

One or more vital sign sensors 112 may be further located in the room 104. The vital sign sensor(s) 112 may be configured to detect one or more vital signs and/or parameters of the patient 106. As used herein, the term “vital sign,” and its equivalents, can refer to a parameter indicating a medical status of a patient. Vital signs include, for example, temperature (e.g., body temperature), pulse rate, respiration rate, blood pressure, airway CO₂ (e.g., EtCO₂), blood oxygenation (e.g., SpO₂), electrocardiogram (ECG), electroencephalogram (EEG), electrolyte (e.g., sodium and potassium) levels, or any combination thereof. Other parameters of the patient 106 include an amount, rate, or frequency of a fluid and/or medication administered to the patient 106. In some cases, the vital sign sensor(s) 112 include a sensor configured to detect a fluid and/or a medication administered to the patient 106. For example, the vital sign sensor(s) 112 may be coupled to and/or incorporated into an intravenous (IV) fluid pump that administers a fluid to the patient 106. The vital sign sensor(s) 112, for example, detect an amount of fluid delivered intravenously to the patient 106 via the IV fluid pump and/or a medication administered to the patient 106 in the fluid.

In some cases, the electronic whiteboard 102 may include and/or be connected to one or more location sensors 114. The location sensor(s) 114 may be located in the room 104, but implementations are not so limited. In various implementations, the location sensor(s) 114 may be configured to detect a location of the patient 106, the visitor 110, or any other individual present in the room 104. In some cases, the location sensor(s) 114 are configured to detect a location of an object (e.g., the vital sign sensor(s) 112) in the room 104.

Although not specifically illustrated in FIG. 1, in some cases, multiple patients may be located in the room 104. For instance, the room 104 may be a dual- or multi-occupancy room. In some cases, each patient within the room 104 is associated with an individual electronic whiteboard 102. The room 104 may include one or more partitions (e.g., curtains) separating the multiple patients. In various implementations, the electronic whiteboard 102 is configured to identify the partition(s) and identify the patient 106 based on the relative location of the patient 106 with respect to the partition(s). In some cases, the electronic whiteboard 102 is configured to identify another electronic whiteboard within the room 104. For instance, the electronic whiteboard 102 may distinguish between the patient 106 and other patients in the room 104 based on the presence of the other electronic whiteboard.

In various cases, the electronic whiteboard 102 may be communicatively connected to one or more electronic medical record (EMR) servers 116 via one or more communication networks 118. The communication network(s) 118 include wired (e.g., electrical or optical) and/or wireless (e.g., radio access, BLUETOOTH, WI-FI, or near-field communication (NFC)) networks. The communication network(s) 118 may forward data in the form of data packets and/or segments between various endpoints, such as computing devices, medical devices, servers, and other networked devices in the environment 100.

The EMR server(s) 116 may store EMRs of multiple patients including the patient 106. As used herein, the terms “electronic medical record,” “EMR,” “electronic health record,” and their equivalents, can refer to a data indicating previous or current medical conditions, diagnostic tests, or treatments of a patient. The EMRs may also be accessible via computing devices operated by care providers. In some cases, data stored in the EMR of a patient is accessible to a user via an application operating on a computing device. For instance, patient data may indicate demographics of a patient, parameters of a patient, vital signs of a patient, notes from one or more medical appointments attended by the patient, medications prescribed or administered to the patient, therapies (e.g., surgeries, outpatient procedures, etc.) administered to the patient, results of diagnostic tests performed on the patient, patient identifying information (e.g., a name, birthdate, etc.), or any combination thereof.

As illustrated, the environment 100 also includes a care provider 120. As used herein, the terms “medical care provider,” “care provider,” and their equivalents, can refer to an individual responsible for monitoring, treating, diagnosing, or managing health care of at least one patient. Examples of care providers include nurses, physicians, physician assistants, therapists (e.g., respiratory therapists, physical therapists, etc.), and medical technicians. As used herein, the terms “health care,” “medical care,” and their equivalents, can refer to diagnostic and/or therapeutic medical interventions performed on a patient. As used herein, the terms “responsible for,” “assigned to,” and their equivalents, can refer to a relationship between one or more patients and a care provider responsible for monitoring and/or caring for the patient(s).

The care provider 104 may wear, carry, or otherwise be attached to a badge 122. The badge, for example, may be an identification (ID) badge 122 of the care provider 120. In some examples, the badge 122 may include a radio frequency identification (RFID) tag. For example, the location sensor(s) 114, in some cases, are configured to detect a location of the badge 122 by transmitting and/or receiving radio frequency (RF) signals with the RFID tag. For example, the location sensor(s) 114 may include a badge reader.

In particular cases, the care provider 120 may further wear, carry, or otherwise be associated with a care provider device 124. The care provider device 124 may be a computing device. For example, the care provider device 124 may be a mobile phone, a tablet computer, a laptop computer, a personal digital assistant (PDA), or some other type of computing device. In some implementations, the care provider 120 may access the EMR of the patient 106 via the care provider device 124. For example, the care provider device 124 may execute an application that enables the care provider device 124 to receive information in the EMR of the patient 106 from the EMR server(s) 116.

In various implementations, the electronic whiteboard 102 may be communicatively coupled to or include the one or more location sensors 114. The location sensor(s) 114 may be configured to identify the location of at least one of the patient 106, the visitor 110, or the care provider 120. In some examples, the location sensor(s) 114 include one or more microphones configured to detect voices of the patient 106, the visitor 110, and the care provider 120. In some cases, the location sensor(s) 114 may be configured to capture audio and identify the voices of the patient 106, the visitor 110, and the care provider 120 within the audio. For instance, the location sensor(s) 114 may perform voice recognition on the captured audio. The location sensor(s) 114, for example, may determine the distances between the location sensor(s) 114 and the patient 106, the visitor 110, and the care provider 120 based on the magnitude (e.g., volume) of the voices detected by the microphone(s). In some instances, the location sensor(s) 114 may include a microphone array configured to detect the voices of the patient 106, the visitor 110, and the care provider 120. The location sensor(s) 114 may be configured to identify the locations of the patient 106, the visitor 110, and the care provider 120 in the environment 100 by applying triangulation techniques to the respective signals detected by the microphones in the array.

In some implementations, the location sensor(s) 114 may include one or more cameras. The camera(s) may be configured to capture images and/or videos of the patient 106, the visitor 110, and the care provider 120. In some examples, the location sensor(s) 114 may be configured to recognize the patient 106, the visitor 110, and the care provider 120 in captured images and/or videos. For example, the location sensor(s) 114 may perform facial recognition on the images and/or videos. In some examples, the location sensor(s) 114 may identify distances between the location sensor(s) 114 and the patient 106, the visitor 110, and the care provider 120 based on a sizes of depictions of the patient 106, the visitor 110, and the care provider 120 in the images and/or videos. According to various implementations, the location sensor(s) 114 may include multiple cameras configured to capture images and/or videos at different locations. The location sensor(s) 114 may identify the locations of the patient 106, the visitor 110, and the care provider 120 within the environment 100 using triangulation techniques. In some implementations, the location sensor(s) 114 identifies the location and/or presence of the patient 106, the visitor 110, or the care provider 120 in the room 104 by identifying a visual symbol displayed on the patient 106, the visitor 110, or the care provider 120. For example, the patient 106, the visitor 110, or the care provider 120 may wear an article (e.g., clothing, a bracelet, a necklace, etc.) that is printed with a QR code or some other visual symbol that can be recognized in an image obtained by camera(s) of the location sensor(s) 114.

According to some examples, the location sensor(s) 114 may include one or more RFID sensors disposed at predetermined positions in the environment 100. In some cases, the location sensor(s) 114 receive wireless signals (e.g., NFC signals, radio frequency RF signals, etc.) from tags (e.g., a tag attached to the patient 106, a tag attached to the support structure 108, a tag attached to the visitor 110, the tag in the badge 122, etc.) located within the environment 100. The location sensor(s) 114 may identify the times at which the wireless signals were received. The location sensor(s) 114 may determine the location of the tags based on the times at which the wireless signals were received. Accordingly, the location sensor(s) 114 may derive the locations of the tags using triangulation. In various implementations, the location sensor(s) 114 may determine the locations of the patient 106, the visitor 110, and the care provider 120 based on the locations of the tags.

In some cases, the location sensor(s) 114 include a badge reader configured to read the badge 122 when the care provider 120 is located in the room 104. For instance, the location sensor(s) 114 may include an optical scanner configured to identify a bar code or QR code printed on the badge 122. The location sensor(s) 114 may determine that the care provider 120 is located in the room 104 and/or near the electronic whiteboard based on identifying the bar code or QR code.

The electronic whiteboard 102 may include one or more input devices 126 and one or more output devices 128. The input device(s) 126 may be configured to receive input signals from the patient 106, the visitor 110, and the care provider 120. For example, the input device(s) 126 may include one or more touch sensors, one or more pressure sensors, one or more cameras (e.g., IR cameras, video cameras, etc.), one or more microphones, one or more keyboards, one or more buttons, one or more accelerometers, one or more gyroscopes, or any combination thereof. In some examples, the location sensor(s) 114 are at least a portion of the input device(s) 126. The output device(s) 128 may be configured to provide output information to the patient 106, the visitor 110, and the care provider 120. For instance, the output device(s) 128 may include one or more display screens (e.g., light emitting diode (LED) screens, organic LED (OLED) screens, liquid crystal display (LCD) screens, etc.), one or more displays, one or more lights, one or more speakers, one or more haptic feedback devices, one or more refreshable braille displays, one or more holographic display devices, or any combination thereof. In various cases, the output device(s) 128 may include a screen 130 configured to visually provide output signals to the patient 106, the visitor 110, and the care provider 120. In some cases, the screen 130 is a touchscreen including one or more touch sensors integrated into a display surface. In various examples, the screen 130 is another type of display, such as a projector screen, a virtual reality (VR) headset, or a holographic display. The electronic whiteboard 102 may include an internal clock and automatically dim or brighten the screen 130 at predetermined times of day. For example, the electronic whiteboard 102 may dim the screen 130 at a time in the evening (e.g., at 9 PM) and may brighten the screen at a time in the morning (e.g., at 9 AM).

In various implementations, the electronic whiteboard 102 may output information related to the patient 106 based on the locations of at least one of the patient 106, the visitor 110, or the care provider 120. For instance, with reference to FIG. 1A, the electronic whiteboard 102 may determine that the patient 106 is in the room 104 and/or within a first threshold distance of the electronic whiteboard 102, the visitor 110 is in the room 104 and/or within a second threshold distance of the electronic whiteboard 102, and the care provider 120 is outside of the room 104 and/or outside of a third threshold distance of the electronic whiteboard 102. Based on these determinations, the electronic whiteboard 102 may output first information associated with the patient 106.

In some implementations, the first information is output as a first graphical user interface (GUI) 132 on the screen 130. As used herein, the terms “graphical user interface,” “GUI,” and their equivalents, may refer to a visual means for outputting information to a user and/or receiving information from the user. In some cases, the first information is output using other output device(s) 128. For example, the first information may be visually output by the one or more lights, audibly output by the one or more speakers, physically output by the one or more haptic feedback devices, as braille on the one or more refreshable braille displays, or as a hologram using the one or more holographic display devices.

The first information may be directed to the patient 106 and/or the visitor 110, rather than the care provider 120. For example, the first information may include non-technical information about the stay of the patient 106 in the room 104 and/or the environment 100. The first information may include a current time and/or date. In some cases, the first information may indicate an identity of the care provider 120. For example, the first information may indicate a name of the care provider 120 and/or a role of the care provider 120 within the environment 100 (e.g., the first information may indicate that the care provider 120 is a CNA, a nurse, a physician's assistant (PA), a physical therapist, a resident, a physician, a medical or nursing student, etc.). In some cases, the first information may include contact information of the care provider 120, such as a phone number and/or information enabling participation in a video conference with the care provider 120. In some cases, the first information may include a pain scale of the patient 106.

In some cases, the first information may include one or more care goals of the patient 106. For example, the first information may include non-technical descriptions of vital sign ranges (e.g., temperature ranges, blood pressure ranges, etc.), ambulation routines, wound healing goals, etc., that the care provider 120 may have set for the patient 106. In some cases, the care goal(s) may include one or more milestones that the patient 106 can achieve before being discharged from the environment 100. By providing the first information to the patient 106 and/or the visitor 110, the patient 106 and/or the visitor 110 may be encouraged to perform actions and/or watch for signs that are associated with achieving the care goal(s).

In various instances, the first information may include one or more patient instructions. The instruction(s), for example, may direct the patient 106 and/or the visitor 110 to perform one or more actions for achieving the care goal(s). For example, the instruction(s) may indicate a diet of the patient 106 (e.g., do not eat within a particular time period before a scheduled surgery, drink a certain number of cups of water in a time period, avoid foods with relatively high salt content, etc.). In some cases, the instruction(s) may direct the patient 106 to perform exercises and/or movements (e.g., walk around the environment 100 once every four hours, turn over every hour, etc.), which may or may not be done with the assistance of the visitor 110. In some examples, the instruction(s) may direct the patient 106 to perform a breathing exercise (e.g., perform deep breathing to reduce blood pressure, use an incentive spirometer, etc.). In various cases, the instruction(s) may direct the patient 106 to perform one or more mindfulness exercises, such as breathing exercises, guided meditation, non-pharmaceutical pain management strategies, and so on. In some cases, the electronic whiteboard 102 may output soothing sights or sounds, such as depictions of coastal scenes, forest scenes, or scenes from a country-of-origin of the patient 106.

According to some examples, the first information may indicate a schedule of the patient 106. For instance, the patient 106 may be scheduled for appointments with the care provider 120, and the time and length of those appointments may be included in the first information. In some cases, the patient 106 may be scheduled for sleep, medication doses, toileting, therapies (e.g., physical therapy, incentive spirometer usage, ambulation, etc.), movement (e.g., changing position in the support structure 108 to avoid pressure injury), meals, and so on, which may be indicated in the first information. Similarly, the first information may include one or more timers that are associated with events that the patient 106 can achieve without assistance from the care provider 120, and which may be in furtherance of the care goal(s) of the patient 106. For example, the first information may include a timer indicating a time until a next scheduled movement event (e.g., turning in the support structure 108, standing up, getting up out of the support structure 108, walking around the environment 100, etc.) associated with avoiding pressure injuries due to immobility in the support structure 108. In some cases, the first information may include a timer for a spontaneous awakening trial (SAT) and/or a spontaneous breathing trial (SBT) of the patient 106, which can promote adherence of the patient 106 to their care goals and to reduce the chance of developing delirium.

In some implementations, the first information may include one or more educational resources. For example, the first information may explain at least one of a condition, a care goal, or a therapy of the patient 106 to a lay audience. In some cases, the first information may include articles, images, and/or videos explaining details about the condition, care goal, and/or therapy. Thus, the patient 106 and/or the visitor 110 may inform themselves about the condition, care goal, and/or therapy, even when the care provider 120 is absent from the room 104. The educational resources may be pre-stored in memory of the electronic whiteboard 102 and/or received from a remote computing device over the communication network(s) 118.

According to some examples, the first information may include one or more care games. The care game(s), for example, may include at least one virtual game configured to achieve the care goal(s) of the patient 106. In some cases, the care game(s) may include gamification of the patient instruction(s) and/or timer(s). For instance, the patient 106 may earn points and/or unlock levels in the care game(s) based on completing tasks, such as ambulation, movement (e.g., number of steps walked), physical therapy exercises, respiratory therapy exercises, and the like. In some cases, the first information may compare the progress of the patient 106 in the game(s) to at least one other patient in the environment 100, to previous patients in the environment 100, and/or to an average patient profile.

In various implementations, the first information may include a voice and/or video conference portal that can connect to a computing device (not illustrated) associated with the care provider 120. For example, if the patient 106 and/or the visitor 110 are concerned about a condition of the patient 106, the patient 106 and/or the visitor 110 may activate the conference portal to establish a communication session between the electronic whiteboard 102 and the device of the care provider 120. In some cases, the conference portal can enable the patient 106 and/or the visitor 110 to communicate with computing devices used by other people with the same condition as the patient 106, family members of people with the same condition as the patient 106, or the like.

Referring to FIG. 1B, in some implementations, the electronic whiteboard 102 may identify, at a second time, that the care provider 120 is within the third threshold distance of the electronic whiteboard 102 and/or within the room 104. Accordingly, the electronic whiteboard 102 may output second information about the patient 106, rather than the first information. The second information may be specifically directed to the care provider 120. For example, the second information may include technical information related to the care of the patient 106 in the environment 100. In some implementations, the electronic whiteboard 102 outputs at least a portion of the second information as a second GUI 134 on the screen 130. In some cases, the electronic whiteboard 102 outputs at least a portion of the second information using the output device(s) 128. For instance, the electronic whiteboard may output the second information visually output by the one or more lights, audibly output by the one or more speakers, physically output by the one or more haptic feedback devices, as braille on the one or more refreshable braille displays, or as a hologram using the one or more holographic display devices.

In some examples, the second information may include at least some similar information to the first information. For instance, the second information may include the care goal(s) and/or pain scale. In various cases, the second information may be at least partially different than the first information. For example, the second information may omit care provider identities and/or contact information, instructions to be followed by the patient, patient-specific timers, care games, educational resources, or any combination thereof.

For instance, the second information may include patient information. The patient information may indicate an identity (e.g., a name and/or image) of the patient 106 and/or an identity (e.g., a name and/or image) of the visitor 110. In some cases, the patient information may indicate a language spoken by the patient 106 and/or visitor 110. According to some examples, the patient information indicates that the patient 106 is visually impaired, and may instruct the care provider 120 to verbally announce themselves as they are entering the room.

The second information may, in some cases, indicate one or more tasks assigned to the care provider 120 and/or at least one timer associated with the task(s). In some cases, the timer(s) may indicate a time since and/or time until toileting, a time since and/or a time since a medication was administered to the patient 106, a time since and/or time until a therapy is performed on the patient 106, or a combination thereof. The timer(s) may count up or down to events. The events, in some cases, can be detected based on parameters detected by the support structure 108 and/or vital signs (or parameters) detected by the vital sign sensor(s) 112.

In some examples, the second information may include diagnostic information. For example, the second information may include one or more vital signs of the patient 106. In some cases, the second information may indicate progression of a wound of the patient 106. In various cases, the second information may include one or more diagnostic images of the patient 106, such as a magnetic resonance imaging (Mill) image, an x-ray image, a computed tomography (CT) image, a positron emission tomography (PET) image, a SPECT image, an ultrasound image and/or video, or any combination thereof.

The second information may indicate one or more conditions and/or statuses of the patient 106. In particular cases, the second information may indicate a level of physical assistance (e.g., 1-person assist or 2-person assist) and/or equipment (e.g., a walker, a wheelchair, etc.) that the patient 106 uses for ambulation. In some cases, the second information may include trends associated with the patient 106, such as ambulation and/or mobility trends of the patient 106 over time. For instance, the second information may indicate that the patient 106 has transitioned from a 2-person assist to a 1-person assist for toileting, which may provide the care provider 120 with additional context about the status of the patient 106. The condition(s) may include a mobility level of the patient 106, a level of assistance required by the patient 106 for completing tasks, a wound progression of the patient 106, labs of the patient 106, a blood sugar level of the patient 106, an intake-output (e.g., a water intake and urine output) of the patient 106, a stroke care progression of the patient 106, WBC of the patient 106, an objective pain level of the patient 106, or a combination thereof.

According to some cases, the condition(s) of the patient 106 may include a risk of the patient 106. For example, the second information may include a falls risk of the patient 106, a sepsis risk of the patient 106, a pressure injury risk of the patient 106, an aspiration risk of the patient 106, or any combination thereof. In some examples, the electronic whiteboard 102 may derive a risk of the patient 106 based on parameters detected by the support structure 108 and/or vital signs (or other parameters) detected by the vital sign sensor(s) 112. The condition(s) may further include one or more precautions associated with treating the patient 106. For example, the second information may indicate a contact precaution, an airborne precaution, a patient aggression precaution, an affinity towards substance abuse (e.g., opioid and/or alcohol abuse), a bloodborne pathogen precaution, an allergy, a bleeding precaution, or any combination thereof. The status(es) of the patient 106 may indicate past and/or future therapies that are performed on the patient 106 (e.g., past or future surgeries) and/or a DNR status of the patient 106.

In various implementations, the second information may include one or more goals of the patient 106. For instance, the second information may include a discharge plan, physical therapy and/or occupational therapy (PT/OT) goals for the patient 106. In some examples, the electronic whiteboard 102 may include a blank screen that can display a drawing or note input by the care provider 120, which can be reviewed by the patient 106, the visitor 110, and the care provider 120 simultaneously. For instance, the care provider 120 may draw a diagram indicating a surgery that the patient 106 will have in the future. Thus, the electronic whiteboard 102 may be used for interactions between the patient 106, the visitor 110, and the care provider 120.

According to some examples, the electronic whiteboard 102 may identify a condition of the patient 106 and adapt the second information based on the condition. In some cases, the electronic whiteboard 102 may detect the patient 106 in cardiac arrest and/or pulmonary arrest. For instance, the electronic whiteboard 102 may identify the condition of the patient 106 based on one or more vital signs and/or parameters detected by the vital sign sensor(s) 112. In emergency conditions like cardiac and/or pulmonary arrest, the electronic whiteboard 102 may restrict the amount of second information output in the room 104, which may limit distractions to the care provider 120. The electronic whiteboard 102 may selectively output information pertinent to treating the identified condition of the patient 106. For example, the electronic whiteboard 102 may output a timer indicating a time since the. For example, the electronic whiteboard 102 may output a timer indicating a time since the cardiac and/or pulmonary arrest was identified, an instruction for treating the patient 106 (e.g., an instruction to administer CPR), a DNR order of the patient 106 (if applicable), allergies of the patient 106 to any medications that may be administered during immediate treatment of the cardiac and/or pulmonary arrest, and so on. In some cases, the instruction for treating the patient 106 may enable the care provider 120, even if the care provider 120 is a novice care provider (e.g., a medical or nursing student), to provide immediate care to the patient 106 until a more experienced code team arrives in the room 104.

The first GUI 132 and the second GUI 134 may be adaptable. For example, the first GUI 132 and the second GUI 134 may include multiple screens and/or UI elements that appear or are hidden based on signals received by the input device(s) 126. That is, the patient 106, the visitor 110, and/or the care provider 120 may navigate the screens of the first GUI 132 and/or the second GUI 134 using the input device(s) 126. Accordingly, each one of the first GUI 132 and the second GUI 134 may be capable of displaying more information than the screen 130 could display at a single time.

Although not illustrated in FIGS. 1A and 1B, in some cases, the environment 100 may include multiple care providers that are responsible for the patient 106. The care providers may have different roles in the care of the patient 106. The electronic whiteboard 102 may be configured to output different information based on the presence of the different care providers and their different roles. For instance, the electronic whiteboard 102 may output a toileting schedule upon identifying that the location of a CNA is within the room 104, because the CNA may be responsible for assisting the patient 106 with toileting. However, the electronic whiteboard 102 may refrain from outputting a diagnostic image (e.g., a PET-MM scan) of the patient 106 to the CNA, because the diagnostic image may be irrelevant to the CNA's duties. However, the electronic whiteboard 102 may output the diagnostic image upon identifying that the location of a surgeon is within the room 104, because the diagnostic image may be relevant to the surgeon's decision of whether to counsel the patient 106 to pursue surgery. However, the electronic whiteboard 102 may refrain from outputting the toileting schedule to the surgeon, because the toileting schedule may be minimally relevant to the surgeon's role in caring for the patient 106.

In particular implementations, the electronic whiteboard 102 may identify the first information and/or the second information based on parameters detected by the support structure 108. For example, the electronic whiteboard 102 may reset a movement timer for reducing a pressure injury risk of the patient 106 based on the support structure 108 detecting parameter(s) indicative of movement of the patient 106. In some cases, the electronic whiteboard 102 may provide points to the patient 106 in the care game based on movement detected by the support structure 108. In some cases, the electronic whiteboard 102 may adjust a risk of the patient 106 (e.g., a falls risk, a pressure injury risk, etc.) based on parameters detected by the support structure 108.

According to some examples, the electronic whiteboard 102 may identify the first information and/or the second information based on the vital sign sensor(s) 112. For example, the second GUI 134 may display updated vital signs as they are detected by the vital sign sensor(s) 112. In some examples, the electronic whiteboard 102 may output an instruction to perform meditation and/or breathing exercises to the patient 106 upon determining that the blood pressure of the patient 106 has exceeded a threshold.

In some cases, the electronic whiteboard 102 may identify the first information based on one or more signals from the EMR server(s) 116. In various implementations, the EMR server(s) 116 may transmit EMR data to the electronic whiteboard 102. The EMR data may include at least a portion of the EMR of the patient 106. In some examples, the EMR server(s) 116 may transmit updated EMR data periodically and/or repeatedly to the electronic whiteboard 102. Accordingly, the first or second information output by the electronic whiteboard 102 may be as up-to-date as the EMR of the patient 106.

In various examples, the electronic whiteboard 102 may identify the first information and/or the second information based on the signals detected by the input device(s) 126. For instance, the care provider 120 may input a drawing of a diagram of a surgery to be performed on the patient 106, and the electronic whiteboard 102 may display the drawing and/or save the drawing for further perusal by the patient 106 at a later time. In some cases, the care provider 120 may speak a command indicating that the patient 106 is now a one-person assist, rather than a two-person assist. The electronic whiteboard 102 may detect the command using the microphone(s) in the input device(s) 126 and automatically update the second GUI 134 accordingly.

According to various implementations, the electronic whiteboard 102 may automatically update the EMR of the patient 106 based on the vital signs and/or parameters detected by the vital sign sensor(s) 112 and/or the signals detected by the input device(s) 126. The electronic whiteboard 102 may generate update data based on the vital signs, parameters, and/or signals detected by the input device(s) 126 and transmit the update data to the EMR server(s) 116. The EMR server(s) 116 may modify the EMR of the patient 106 based on the update data.

According to some cases, the electronic whiteboard 102 may detect writing of the patient 106, the visitor 110, and/or the care provider 120. For example, the screen 130 may be a touchscreen and the electronic whiteboard 102 may detect a word written on the touchscreen in accordance with a touch signal detected by the touchscreen. In some cases, the input device(s) 126 include one or more touch sensors that are separate from the screen 130, which can detect a touch signal indicative of a written word. In some implementations, a camera in the input device(s) 126 may capture an image and/or video of a word written on a substrate (e.g., a piece of paper) by the patient 106, the visitor 110, or the care provider 120. In various implementations, the electronic whiteboard 102 may perform optical character recognition (OCR) on signals detected by the input device(s) 126 in order to identify written words. In various implementations, the electronic whiteboard 102 may update the EMR of the patient 106 based on the words. For example, the electronic whiteboard 102 may generate update data based on the written words.

In some cases, the electronic whiteboard 102 may generate update data based on one or more commands spoken by the care provider 120. The care provider 120 may speak the command(s) in the room 104 and the electronic whiteboard 102 may detect the command(s) using one or more microphones in the input device(s) 126. For example, the command(s) may indicate a diagnostic finding of the patient 106 (e.g., an arrhythmia of the patient 106 observed by the care provider 120 in the room), a therapeutic finding of the patient 106 (e.g., an order by the care provider 120 for a medication to be administered to the patient 106 or a change in an amount or scheduling of the medication), a condition and/or status of the patient 106 (e.g., a change from a two-person assist to a one-person assist), or any combination thereof. In some cases, the command(s) may include words spoken by the care provider 120 to the patient 106 and/or visitor 110, such as instructions for achieving a care goal of the patient 106. The electronic whiteboard 102 may use natural language processing to identify words spoken in the command(s).

The electronic whiteboard 102 may cause the EMR server(s) 116 to automatically update the EMR of the patient 106 based on the update data. Accordingly, the EMR may be updated without the care provider 120 logging into or manually typing changes to the EMR of the patient 106 into a separate computing device. In some implementations, the command(s) are spoken as the care provider 120 is treating the patient 106. For example, the care provider 120 may speak the command(s) while the care provider 120 is administering CPR to the patient 106. Thus, the electronic whiteboard 102 may enable the care provider 120 to update the EMR of the patient 106 even when the hands of the care provider 120 are occupied with other tasks.

By providing a convenient mechanism for updating the EMR of the patient 106, even while the care provider 120 is actively caring for the patient 106, the electronic whiteboard 102 may reduce or even eliminate the risk that medically relevant information about the patient 106 will not be documented in the EMR. Furthermore, because the electronic whiteboard 102 to accurately update the EMR of the patient 106 without requiring the care provider 120 to log in or activate a separate computing device, the electronic whiteboard 102 may simplify EMR documentation for the patient 106, thereby enabling the care provider 120 to spend more time on patient care and less time on administrative tasks. In addition, these features enable the electronic whiteboard 102 to provide accurate communication about the patient 106 between different care providers in the environment 100.

The electronic whiteboard 102 may adjust size of text and/or icons in the first GUI 132 and/or the second GUI 104 based on the locations of the patient 106, the visitor 110, and/or the care provider 120. For example, the electronic whiteboard 102 may identify a distance between the electronic whiteboard 102 and a viewer (e.g., the patient 106, the visitor 110, or the care provider 120). The electronic whiteboard 102 may adjust a font size of text displayed on the screen 130 based on the distance. For example, the electronic whiteboard 102 may cause the font size to be proportional and/or positively correlated to the distance between the electronic whiteboard 102 and the viewer. Accordingly, the text on the electronic whiteboard 102 may be readable by the viewer, even if the electronic whiteboard 102 is relatively far from the viewer in the viewer's line-of-sight. In some cases, the electronic whiteboard 102 may adjust the font size of text intended for the patient 106 based on an indication of an ophthalmic health state and/or visual acuity of the patient 106 in the EMR of the patient 106. For example, if the EMR indicates that the patient 106 has macular degeneration, the electronic whiteboard 102 may output the first information at a relatively large font size.

In some cases, the electronic whiteboard 102 may adjust the volume of output signals indicating the first information and/or the second information based on the locations of the patient 106, the visitor 110, and/or the care provider 120. For example, the electronic whiteboard 102 may identify a distance between the electronic whiteboard 102 and a listener (e.g., the patient 106, the visitor 110, or the care provider 120). The electronic whiteboard 102 may adjust the volume of auditory signals output by one or more speakers of the output device(s) 128 based on the distance. For instance, the electronic whiteboard 102 may output an auditory signal at a volume that is proportional and/or positively correlated to the distance between the electronic whiteboard 102 and the listener. In some cases, the electronic whiteboard 102 may adjust the volume based on the EMR of the patient 106. For example, if the EMR indicates that the patient 106 is hard-of-hearing, the electronic whiteboard 102 may automatically output the first information at a relatively high volume.

In some examples, the electronic whiteboard 102 may adjust a language of the first information and/or the second information based on the input device(s) 126. In some examples, the input device(s) 126 may detect a signal indicative of a language of a user (e.g., the patient, the visitor 110, or the care provider 120). For instance, a microphone may detect words spoken by the user, a touchscreen and/or camera may detect words written by the user, or any combination thereof. In various implementations, the electronic whiteboard 102 may apply natural language processing to the words. In addition, the electronic whiteboard 102 may identify a language of the words, for example, by comparing the words to one or more dictionaries stored or otherwise accessible by the electronic whiteboard 102. In various implementations, the electronic whiteboard 102 may automatically adjust words output by the output device(s) 128 and/or screen 130 to be in the identified language. For example, if the patient 106 and the visitor 110 are speaking Tagalog, the electronic whiteboard 102 may identify that the patient 106 and the visitor 110 are speaking Tagalog and may automatically output the first information in Tagalog. Accordingly, the electronic whiteboard 102 may communicate relevant information to the users in their native languages. In some cases, the electronic whiteboard 102 may translate words input by the care provider 120 into the language preferred by the patient 106 and/or visitor 110, or vice versa. Thus, the electronic whiteboard 102 may provide translation services.

In some instances, the electronic whiteboard 102 may translate technical terms and/or medical jargon. For example, the input device(s) 126 may identify a word input by the care provider 120 (e.g., spoken by the care provider 120, written by the care provider 120, etc.). The electronic whiteboard 102 may compare the word to a jargon dictionary, which may be stored by the care provider 120. If the electronic whiteboard 102 determines that the word is listed in the jargon dictionary, the electronic whiteboard 102 may translate the jargon term into a non-jargon term. For example, if the whiteboard 102 identifies the term “DX” in written notes of the care provider 120, the term “DX” may be listed in the jargon dictionary as referring to “diagnosis.” Rather than storing the term “DX” in the EMR or outputting the term “DX” on the screen 130 to the patient 106 and/or visitor 110, the electronic whiteboard 102 may store the term “diagnosis” in the EMR or output the term “diagnosis” on the screen.

The electronic whiteboard 102, in some instances, may automatically adjust a brightness of the screen 130 based on the time-of-day. In some cases, the screen 130 may have a first brightness during a first time-of-day and a second brightness during a second time-of-day. For example, the electronic whiteboard 102 may have a high brightness at noon and a low brightness at midnight.

The electronic whiteboard 102, in some cases, can be controlled in a hands-free manner. For example, in some implementations, the input device(s) 126 may exclusively include noncontact devices, such as microphones and/or cameras. In some cases, the signals detected by the input device(s) 126 may exclusively include auditory signals (e.g., spoken words) and/or visual signals (e.g., images of writing). For example, the electronic whiteboard 102 may omit buttons, touch sensors, and other types of input devices that detect physical manipulation. Thus, the electronic whiteboard 102 may adjust and/or output information, and update the EMR of the patient 106, without users touching the electronic whiteboard 102. Accordingly, the electronic whiteboard 102 can limit cross-contamination within the environment 100.

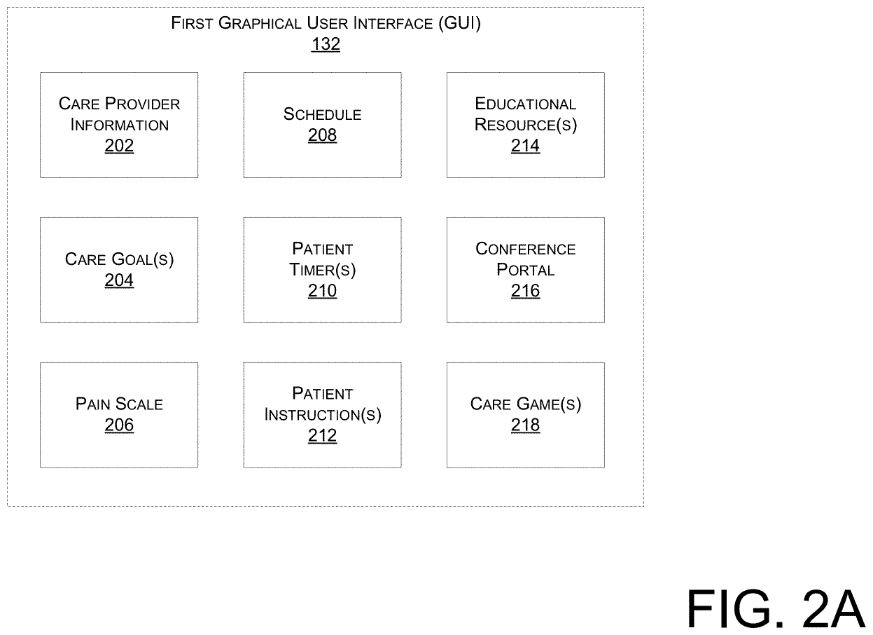

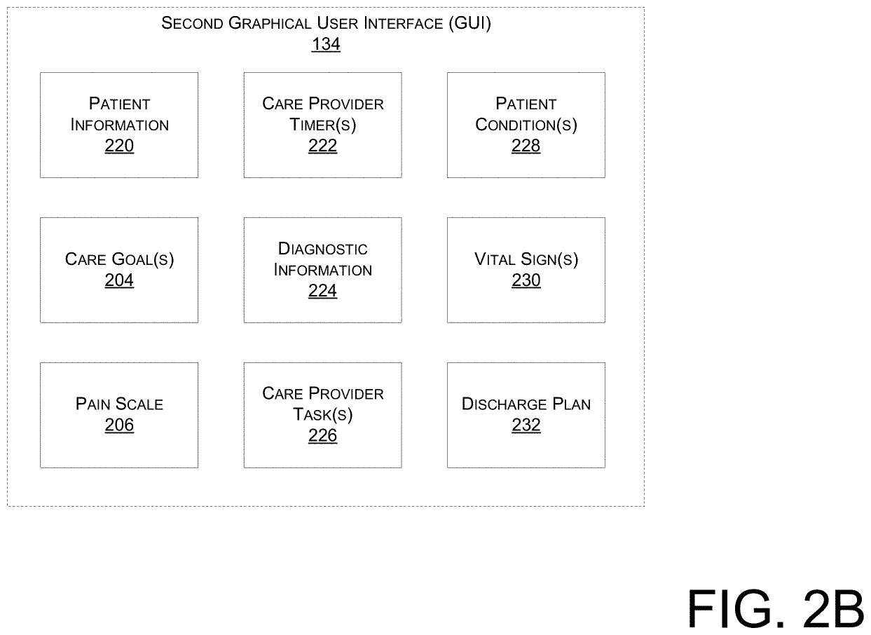

FIGS. 2A and 2B illustrate examples of different GUIs that may be output by the screen 130 of the electronic whiteboard 102. FIG. 2A illustrates an example of the first GUI 132 described above with reference to FIG. 1A. The first GUI 132 may be output and directed to the patient 106 and/or the visitor 110, rather than the care provider 120. In various implementations, the first GUI 132 may include one or more user interface elements. For example, the first GUI 132 may include elements indicating care provider information 202, one or more care goals 204, a pain scale 206, a schedule 208, one or more patient timers 210, one or more patient instructions 212, one or more educational resources 214, a conference portal 216, and one or more care games 218.

The care provider information 202 may inform a user (e.g., the patient 106 and/or visitor 110) about one or more care providers caring for the patient 106 in the clinical environment. In some examples, the care provider 202 lists names of different care providers, roles of different care providers (e.g., physical therapist, respiratory therapist, nurse, physician, resident, etc.), pictures of different care providers, and other identifying information about the care providers. In some implementations, the care provider information 202 may further include contact information for the care providers. For instance, the care provider information 202 may include phone numbers of the care providers, instructions for contacting the care providers over video conferencing, instructions for paging the care providers, or other information indicating how the care providers may be contacted.

The care goal(s) 204 may inform the user about one or more health milestones in furtherance of a recovery of the patient 106 from a medical problem. In some cases, the care goal(s) 204 may indicate one or more milestones before the patient 106 is discharged from the clinical environment. The care goal(s) 204 may indicate ranges of vital signs and/or other parameters. For instance, the care goal(s) 204 may indicate that the patient 106 may be discharged if a core temperature of the patient 106 is within a particular range that is not associated with a fever, or if an input/output of the patient 106 is within a range indicative of healthy kidney function. In some cases, the care goal(s) indicate stages in a wound healing progression. For example, the care goal(s) may indicate that the patient 106 is on their way to recovery if a surgical incision is healed to a threshold extent. In various implementations, the care goal(s) 204 are indicative of actions that the patient 106 may take. For instance, the care goal(s) 204 may include mobility exercises (e.g., walk a loop around the clinical environment once every four hours), respiratory exercises (e.g., use a spirometer once every hour), eating (e.g., consume solid food without vomiting), or any combination thereof.

The pain scale 206 may indicate a level of acute discomfort felt by the patient 106. The patient 106 may self-report their pain level. In some cases, the pain scale 206 may indicate a number along a numeric scale, such as a number between 0 (no pain) and 10 (severe pain). In some implementations, the pain scale 206 may indicate a face along a categorical scale that includes multiple different faces, such as a smiley face (no pain) and a crying face (severe pain). In various implementations, the electronic whiteboard 102 may prompt the patient 106 to self-report their pain level multiple times, such as periodically (e.g., once every hour, once every four hours, etc.). Accordingly, the pain scale 206 may output the most recent pain level self-reported by the patient 106.

The schedule 208 may indicate one or more planned events associated with the patient 106. The schedule 208 may indicate a time-of-day that the events are scheduled and/or a time until the events are scheduled. In some examples, the schedule 208 may indicate medical-related events, such as appointments with care providers (e.g., a checkup with a pulmonologist), diagnostic appointments (e.g., a scheduled MRI scan, a planned cardiac stress test, etc.), therapeutic appointments (e.g., a scheduled surgery, scheduled medications, etc.), and so on. In some implementations, the schedule 208 may indicate non-medical events, such as toileting appointments, meals, sleep events, and the like.

The patient timer(s) 210 may indicate times associated with events that the patient 106 and/or the visitor 110 at least partially control. In some cases, the patient 106 may be assigned to complete particular tasks in order to achieve a care goal indicated in the care goal(s) 204. The patient timer(s) 210 may indicate when the tasks are to be completed. For example, the patient 106 may be assigned to perform a mobility exercise (e.g., walking around the clinical environment), a respiratory exercise (e.g., use of a spirometer), or some other exercise, at a particular frequency (e.g., once every hour, once every four hours, once every eight hours, etc.). The patient timer(s) 210 may indicate a time since the task was performed and/or a time until the next task is to be performed. In particular examples, the electronic whiteboard 102 may output an alert when the patient timer(s) 210 expire, such as a flashing light and/or audible sound.

The patient instruction(s) 212 may direct the patient to perform one or more steps or tasks in furtherance of the patient care goal(s) 204. The patient instruction(s) 212 may indicate tasks that the patient can perform themselves, without assistance from a care provider. In some examples, the patient instruction(s) 212 may direct the patient to perform exercises, such as mobility exercises (e.g., physical therapy exercises, movements, walking, etc.) and/or respiratory exercises (e.g., spirometer exercises). In some examples, the patient instruction(s) 212 include directions to conform to a particular diet and/or sleep schedule. In some cases, the patient instruction(s) 212 may further indicate a schedule for the steps or tasks.

The educational resource(s) 214 may include information about one or more conditions of the patient and/or the patient's care goals. In some cases, the educational resource(s) 214 may include articles, videos, and other informational materials for individuals without specialized medical training. The patient may browse the educational resource(s) 214 for information about their condition(s) and/or care goal(s) at their leisure, even when care providers are not present.

The conference portal 216 may include an icon, application, or other mechanism by which the patient (or a visitor of the patient) may initiate a communication session with a care provider's device. In some cases, the care provider's device is a pager, a tablet computer, a mobile phone, a desktop computer, or some other type of computing device. For example, the communication session may be a voice session, a video conference, a text exchange, or some other type of bidirectional exchange of data between the electronic whiteboard and the provider's device. In some cases, the patient may be concerned about their condition and may contact the care provider via the conference portal 216. The conference portal 216 may display text and/or video of the care provider, who may actively communicate with the patient and/or visitor in the communication session.

The care game(s) 218, in various cases, may include one or more interactive applications through which the patient is encouraged to complete tasks in furtherance of the care goal(s) 204. For example, the patient may earn points or other virtual rewards in response for completing the tasks. In some cases, the care game(s) 218 may compare a progress of the patient to the progress of other patients being cared for in the clinical environment, to the progress of an idealized patient on track for discharge, etc. Thus, the patient's progress may be gamified, thereby providing additional motivation for the patient to achieve the care goal(s) 204.

In various cases, at least one of the care provider information 202, the care goal(s) 204, the pain scale 206, the schedule 208, the patient timer(s) 210, the patient instruction(s) 212, the educational resource(s) 214, the conference portal 216, or the care game(s) 218 may be displayed simultaneously on the screen 130 of the electronic whiteboard 102. For example, the screen 130 may exclusively display the educational resource(s) 214 at one time, may exclusively display the patient timer(s) 210 and care game(s) 218 at another time, and so on. In various implementations, the care provider information 202, the care goal(s) 204, the pain scale 206, the schedule 208, the patient timer(s) 210, the patient instruction(s) 212, the educational resource(s) 214, the conference portal 216, or the care game(s) 218 may include text in a language of the patient and/or visitor.

FIG. 2B illustrates an example of the second GUI 134 described above with reference to FIG. 1B. The second GUI 134 may be output and directed to the care provider 120, rather than the patient 106 and/or the visitor 110. In various implementations, the second GUI 134 may include one or more user interface elements. For example, the second GUI 134 may include patient information 220, at least one care provider timer 222, diagnostic information 224, at least one care provider task 226, one or more patient conditions 228, one or more vital signs 230, and a discharge plan 232. In addition, the second GUI 134 may include one or more user interface elements included in the first GUI 132, such as the care goal(s) 204 and pain scale 206.

The patient information 220 identifies details about the patient. For example, the patient information 220 may include a name of the patient, preferred pronouns of the patient (e.g., she/her/hers or he/him/his), a preferred language of the patient, family members of the patient, and so on. In some cases, the patient information 220 can include details about the patient that other care providers have noted in the EMR of the patient, such as allergies of the patient, whether the patient is under opioid restrictions, etc. According to some implementations, the patient information 220 can indicate whether the patient has consented to procedures, whether the patient has a DNR order, or the like. In some implementations, the patient information 220 indicates that the patient is visually impaired and/or has a hearing deficit. For example, the patient information 220 may instruct the care provider to announce themselves verbally as the enter a room of the patient and/or to make sure to enter the line-of-sight of the patient to visually announce their presence. Thus, the patient information 220 may provide details that can efficiently remind the care provider about the patient upon entering the patient's room.

The care provider timer(s) 222 may indicate times associated with tasks associated with the care provider. In some cases, the care provider may be assigned to complete particular tasks in order to achieve a care goal indicated in the care goal(s) 204. The care provider timer(s) 222 may indicate when the tasks are to be completed. For example, the care provider may be assigned to administer a medication to the patient, to administer another type of therapy to the patient, to assist the patient with toileting, to check vital signs or other health-related metrics of the patient, to assist the patient with performing exercises (e.g., physical therapy exercises, other types of movement, respiratory exercises, speech therapy exercises, etc.), and so on. The care provider timer(s) 222 may indicate a time since the task was performed and/or a time until the next task is to be performed. In particular examples, the electronic whiteboard 102 may output an alert when the care provider timer(s) 222 expire, such as a flashing light and/or audible sound.

The diagnostic information 224 may indicate details about at least one diagnostic test and/or exam of the patient. In some implementations, the diagnostic information 224 may include results of blood tests, medical imaging, and/or the results of other diagnostic tests. In some examples, the diagnostic information 224 may include medical images and/or videos, such as CT scans, MRI images, ultrasound images, plain film X-rays, etc.

The care provider task(s) 226 may indicate one or more instructions for tasks assigned to the care provider. For example, the care provider task(s) 226 may direct the care provider to administer a medication to the patient, to administer another type of therapy to the patient, to assist the patient with toileting, to check vital signs or other health-related metrics of the patient, to assist the patient with performing exercises (e.g., physical therapy exercises, other types of movement, respiratory exercises, speech therapy exercises, etc.), and so on.

The patient condition(s) 228 may indicate one or more conditions of the patient. In some cases, the patient condition(s) 228 may include diagnosed conditions. For instance, the patient condition(s) 228 may indicate that the patient has appendicitis, that the patient has a fever, that the patient is recovering from a surgery, etc. In some examples, the patient condition(s) 228 may indicate why the patient is in the clinical environment. For instance, the patient condition(s) 228 may indicate that the patient is recovering from a surgery, that the patient presented in an emergency department with one or more reported symptoms (e.g., chest pain), etc.

The vital sign(s) 230 may indicate one or more physiological parameters of the patient, such as a body temperature of the patient, a pulse rate of the patient, a respiration rate of the patient, a blood pressure of the patient, an oxygenation level of the patient's blood, an amount of carbon dioxide in the patient's expired breath, any other patient metric that can be monitored and is relevant to the patient's condition, or a combination thereof. In some cases, the vital sign(s) 230 are updated periodically and/or substantially in real time based on one or more vital sign monitors actively monitoring the patient. The care provider may refer to the vital sign(s) 230 in order to assess the condition of the patient.

The discharge plan 232 may indicate one or more patient-related milestones to be achieved prior to discharging the patient from the clinical environment and/or one or more strategies for managing ongoing conditions of the patient after the patient is discharged. According to some examples, the discharge plan 232 may indicate a specified stage of wound care progression, a predetermined pain level, a combination of vital signs, etc., that indicate when the patient can be safely discharged from the clinical environment. In some examples, the discharge plan 232 may include text, images, and/or videos that indicate follow-up instructions, appointments, medications, therapies, dietary guidance, or any combination thereof, for the patient after discharge.

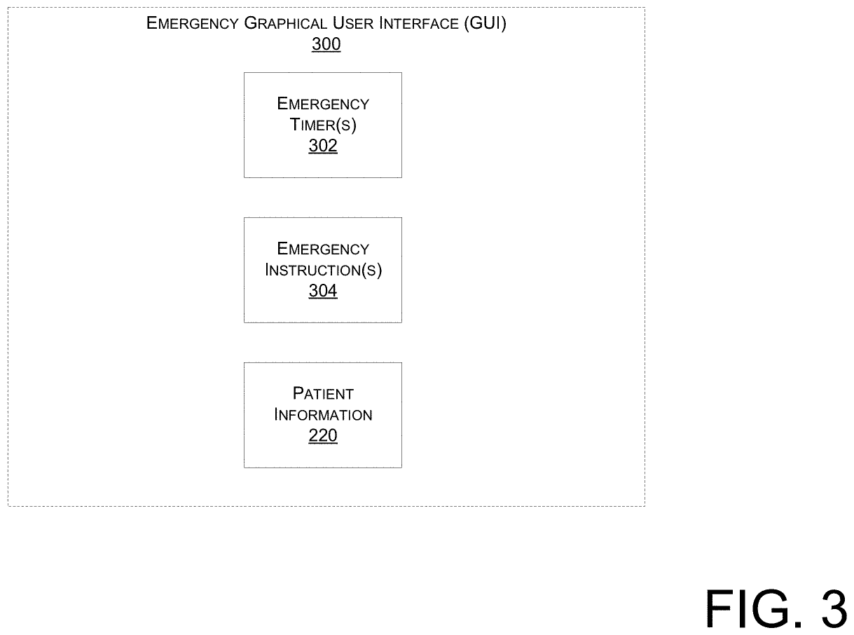

FIG. 3 illustrates an example of an emergency GUI 300 that can be output by the electronic whiteboard 102 described above with reference to FIGS. 1A and 1B. In various implementations, the emergency GUI 300 may be output when the electronic whiteboard 102 detects a medical emergency associated with the patient 106. For example, the electronic whiteboard 102 may output the emergency GUI 300 based on identifying that the patient 106 is in cardiac and/or respiratory arrest based on vital sign(s) detected by the vital sign sensor(s) 112. The emergency GUI 300 may guide a viewer on treating the emergency condition. In some implementations, the emergency GUI 300 is directed to the care provider 120.

The emergency GUI 300 may be simpler than the first GUI 132 and the second GUI 134 described above. For example, the emergency GUI 300 may output only the most pertinent information for addressing the emergency and may omit information associated with long term care of the patient, such as toileting schedules. In particular cases, the emergency GUI 300 may include at least a portion of the patient information 220 described above with reference to FIG. 2B. For instance, the emergency GUI 300 may indicate whether the patient is associated with a DNR order.

The emergency GUI 300 may further include one or more emergency timers 302. The emergency timer(s) 302 may indicate times associated with the emergency and/or treating the emergency. For example, the emergency timer(s) 302 may indicate a time since the emergency was detected. In some examples, the emergency timer(s) 302 may indicate a time associated with a treatment, such as administering a medication to treat the patient.

In addition, the emergency GUI 300 may include one or more emergency instructions 304. The emergency instruction(s) 304 may direct the viewer to perform one or more actions for addressing the emergency. For instance, the emergency instruction(s) 304 may include a direction to administer CPR to the patient, to defibrillate the patient, to administer an emergency medication to the patient, and so on. Accordingly, the electronic whiteboard 102 can support care providers in treating emergencies of patients.

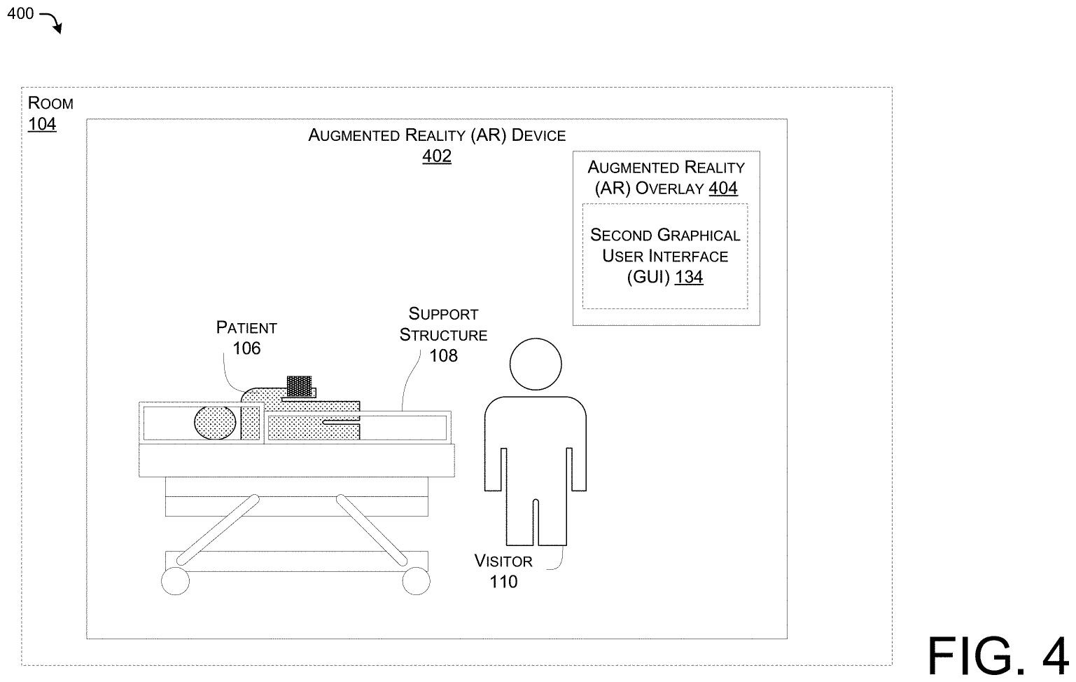

FIG. 4 illustrates an example environment 400 in which an augmented reality (AR) device 402 is used to provide patient-relevant information to a care provider. The AR device 402 may be used as an alternative to the electronic whiteboard 102, in some cases.

In various implementations, the AR device 402 may be worn by a care provider. The AR device 204 may, in some implementations, include a transparent substrate through which the care provider may view the room 104. In some examples, the AR device 402 is an AR headset, such as smart glasses (e.g., GOOGLE GLASS).

The AR device 402, in various implementations, may output an AR overlay 404 on the transparent substrate. From the perspective of the care provider, the AR overlay 404 may take the place of a physical electronic whiteboard screen in the room 104. The AR overlay 404, in various implementations, may include the second GUI 134 described above. Unlike the electronic whiteboard 102 described above, the AR overlay 404 is output to the care provider without being discernible to the patient 106 and/or the visitor 110. Accordingly, the AR overlay 404 may maintain confidentiality of conditions of the patient 106 and/or may display notes that are not appropriate for the patient 106 or the visitor 110. For example, the AR overlay 404 may display sensitive information, such as whether the patient 106 is suffering from opioid dependence, without displaying that sensitive information to the visitor 110.

In some cases, the AR device 402 includes a sensor, or communicates with a device, that detects that the AR device 402 is located in the room 104 with the patient 106. Upon detecting that the AR device 402 is in the room 104, the AR device 402 may output the AR overlay 404 with the second GUI 134 that is specific to the patient 106. Accordingly, the AR device 402 may refrain from outputting information about a patient that is not located in the room 104, while the AR device 402 is located in the room 104.

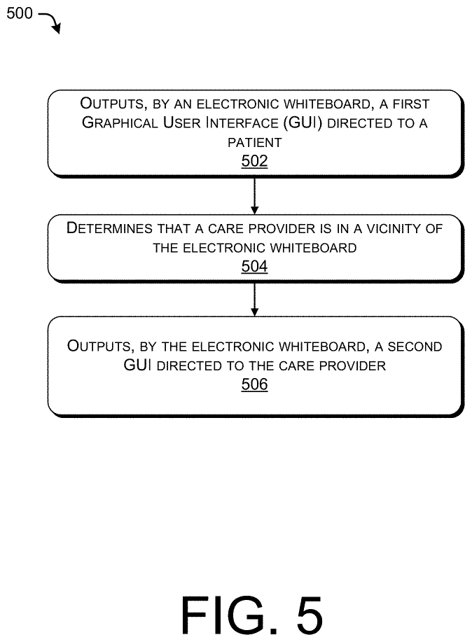

FIG. 5 illustrates an example process 500 for outputting patient information on an electronic whiteboard. In various implementations, the process 500 is performed by an entity, such as a computing device or the electronic whiteboard 102 described above.

At 502, the entity outputs, by an electronic whiteboard, a first GUI associated with a patient. The electronic whiteboard may include a screen that outputs the first GUI. In some implementations, the first GUI includes text. The entity may identify a language of the patient and output the text in the first GUI in the identified language. In some cases, the entity identifies a relative position (e.g., a distance) between the electronic whiteboard and the patient and adjusts a font size of the text based on the distance.

The first GUI may output information relevant to the patient. For example, the first GUI may indicate at least one of an identity of the care provider, contact information associated with the care provider, an ambulation instruction, educational materials about a condition of the patient, a care schedule of the patient, or a game related to the condition of the patient.

At 504, the entity determines that a care provider is in a vicinity of the electronic whiteboard. For example, the entity may determine that the care provider is within a threshold distance of the electronic whiteboard or may determine that the care provider is within the same room as the electronic whiteboard. In various implementations, the entity may include at least one sensor configured to detect the position of the care provider. For example, the entity may include at least one sensor comprises at least one of a microphone array configured to detect a voice of the care provider, an RTLS sensor configured to detect a badge worn by the care provider, or a camera configured to detect an image of the care provider.

At 506, the entity outputs, by the electronic whiteboard, a second GUI directed to the care provider. The second GUI may be different than the first GUI. According to some examples, the second GUI includes text. The entity may identify a language of the care provider and output the text in the second GUI in the identified language. In some implementations, the entity identifies a relative position (e.g., a distance) between the electronic whiteboard and the care provider and adjusts a font size of the text based on the distance.

The second GUI may indicate information pertinent to the care provider. For example, the second GUI may indicate at least one of at least one of contact information of the patient, one or more care goals of the patient, a pain scale of the patient, one or more timers, diagnostic information about the patient, one or more tasks to be completed by the care provider, one or more conditions of the patient, one or more vital signs of the patient, or a discharge plan of the patient. In some cases, the second GUI includes one or more UI elements indicating at least a portion of an EMR of the patient. For example, the entity may receive the EMR of the patient from one or more EMR servers.

In some examples, the entity receives an input signal from the patient or the care provider and updates an EMR of the patient based on the input signal. For example, the entity may detect a voice from the care provider indicating that the care provider is performing a treatment on the patient. The entity may transmit a signal to one or more EMR servers based on the treatment, thereby automatically updating the patient's EMR based on the treatment. Thus, the care provider may update the EMR of the patient as the treatment is being performed and in a hands-free manner.

In some cases, the entity performs quality control on input signals before updating the patient's EMR. The entity may receive, in a UI element, a signal indicative of text. For example, a UI element may include a box that receives information about a patient's name from a user, such as the care provider. The entity may confirm that the text is consistent with the type of UI element. For example, the entity may confirm that the text is a name, rather than a date. If the text, for instance, indicates a number or symbols that are not generally consistent with a name, then the entity may prompt the care provider to confirm that the input signal is correct prior to updating the patient's EMR with the text.

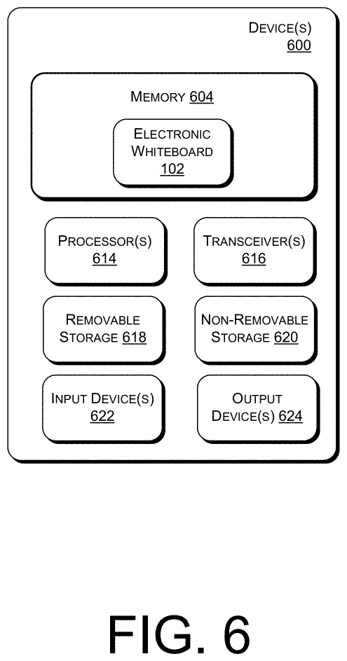

FIG. 6 illustrates at least one example device 600 configured to enable and/or perform the some or all of the functionality discussed herein. Further, the device(s) 600 can be implemented as one or more server computers 602, a network element on a dedicated hardware, as a software instance running on a dedicated hardware, or as a virtualized function instantiated on an appropriate platform, such as a cloud infrastructure, and the like. It is to be understood in the context of this disclosure that the device(s) 600 can be implemented as a single device or as a plurality of devices with components and data distributed among them.

As illustrated, the device(s) 600 comprise a memory 604. In various embodiments, the memory 604 is volatile (including a component such as Random Access Memory (RAM)), non-volatile (including a component such as Read Only Memory (ROM), flash memory, etc.) or some combination of the two.

The memory 604 may include various components, such as instructions for performing operations of the electronic whiteboard 102. The instructions may comprise methods, threads, processes, applications, or any other sort of executable instructions. The instructions can also include files and databases. The instructions can be executed by at least one processor 614 to perform operations. In some embodiments, the processor(s) 614 includes a Central Processing Unit (CPU), a Graphics Processing Unit (GPU), or both CPU and GPU, or other processing unit or component known in the art.

The device(s) 600 can also include additional data storage devices (removable and/or non-removable) such as, for example, magnetic disks, optical disks, or tape. Such additional storage is illustrated in FIG. 6 by removable storage 618 and non-removable storage 620. Tangible computer-readable media can include volatile and nonvolatile, removable and non-removable media implemented in any method or technology for storage of information, such as computer readable instructions, data structures, program modules, or other data. The memory 604, removable storage 618, and non-removable storage 620 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 600. Any such tangible computer-readable media can be part of the device(s) 600.

The device(s) 600 also can include input device(s) 622, such as a keypad, a cursor control, a touch-sensitive display, voice input device, etc., and output device(s) 624 such as a display, speakers, printers, etc. These devices are well known in the art and need not be discussed at length here. In particular implementations, a user can provide input to the device(s) 500 via a user interface associated with the input device(s) 622 and/or the output device(s) 624.

As illustrated in FIG. 6, the device(s) 600 can also include one or more wired or wireless transceiver(s) 616. For example, the transceiver(s) 616 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 616 can utilize Multiple-Input/Multiple-Output (MIMO) technology. The transceiver(s) 616 can include any sort of wireless transceivers capable of engaging in wireless, Radio Frequency (RF) communication. The transceiver(s) 616 can also include other wireless modems, such as a modem for engaging in Wi-Fi, WiMAX, Bluetooth, or infrared communication. In some implementations, the transceiver(s) 616 can be used to communicate between various functions, components, modules, or the like, that are included in the device(s) 600.

### EXAMPLE CLAUSES

1. An electronic whiteboard, including: a screen physically mounted in a room associated with a patient; at least one processor communicatively coupled to the screen; at least one sensor communicatively coupled to the at least one processor and configured to detect a position of a care provider; memory communicatively coupled to the at least one processor and storing instructions that, when executed by the at least one processor, cause the at least one processor to perform operations including: causing the screen to output a first graphical user interface (GUI) associated with the patient; determining that the position of the care provider is at least one of within a threshold distance of the screen or within the room associated with the patient; based on determining that the position of the care provider is at least one of within the threshold distance of the screen or within the room associated with the patient, causing the screen to output a second GUI associated with the care provider, the second GUI being different than the first GUI.
2. The electronic whiteboard of clause 1, wherein the at least one sensor includes at least one of a microphone array configured to detect a voice of the care provider, a real time location system (RTLS) sensor configured to detect a badge worn by the care provider, a radio frequency identification (RFID) reader configured to detect the badge worn by the care provider, or a camera configured to detect an image of the care provider and/or a visual symbol attached to the care provider.
3. The electronic whiteboard of clause 1 or 2, wherein the first GUI includes at least one of an identity of the care provider, contact information associated with the care provider, an ambulation instruction, educational materials about a condition of the patient, a care schedule of the patient, or a game related to the condition of the patient.
4. The electronic whiteboard of any one of clauses 1 to 3, wherein the second GUI indicates at least one of a diagnostic image of the patient, one or more care goals of the patient, a timer related to a condition of the patient, one or more medications prescribed to the patient, one or more treatments performed on the patient, one or more vital signs of the patient, one or more allergies of the patient, or a do-not-resuscitate (DNR) order of the patient.
5. The electronic whiteboard of any one of clauses 1 to 4, further including: a transceiver configured to: receive, from one or more electronic medical record (EMR) servers, EMR data associated with the patient, wherein the first GUI includes one or more first user interface (UI) elements indicating at least a first portion of the EMR data and the second GUI includes one or more second UI elements indicating at least a second portion of the EMR data.
6. The electronic whiteboard of any one of clauses 1 to 5, further including: an input device configured to detect an input signal from at least one of the patient or the care provider; and a transceiver configured to transmit, to one or more electronic medical record (EMR) servers, update data based on the input signal, the one or more EMR servers being configured to modify an EMR associated with the patient based on the update data.
7. The electronic whiteboard of clause 6, wherein the input device includes at least one of a microphone, a touchscreen, or a camera.
8. The electronic whiteboard of clause 6 or 7, wherein: the second GUI includes a UI element corresponding to a predetermined type of information, the input signal indicates one or more words associated with the UI element, and the operations further include: determining that the one or more words are consistent with the type of the information corresponding to the UI element; and based on determining that the one or more words are consistent with the predetermined type of information corresponding to the UI element, causing the transceiver to transmit the update data.
9. The electronic whiteboard of any one of clauses 6 to 8, the care provider being a first care provider, wherein: the at least one sensor is further configured to detect a position of a second care provider, the operations further include: determining that the position of the second care provider is at least one of within the threshold distance of the screen or within the room associated with the patient; based on determining that the position of the second care provider is at least one of within the threshold distance of the screen or within the room associated with the patient, causing the screen to output a third GUI associated with the second care provider, the third GUI being different than the first GUI and the second GUI.
10. A system, including: an output device associated with a patient or a care provider; at least one processor communicatively coupled to the output device; memory communicatively coupled to the at least one processor and storing instructions that, when executed by the at least one processor, cause the at least one processor to perform operations including: causing the output device to output first information about the patient; identifying a condition of the patient; based on identifying the condition of the patient, causing the output device to output second information about the patient, the second information being different than the first information and including at least one of a timer or an instruction for treating the condition of the patient.
11. The system of clause 10, further including: at least one sensor communicatively coupled to the at least one processor and configured to detect a position of the output device, wherein: the output device includes an augmented reality (AR) device worn by the care provider and configured to output the first information and the second information in at least one AR overlay; and the operations further include: determining that the position of the output device is at least one of within a threshold distance of the patient or within a room associated with the patient; and based on determining that the position of the output device is at least one of within the threshold distance of the patient or the room associated with the patient, causing the output device to output the at least one AR overlay.
12. The system of clause 10 or 11, further including: at least one sensor communicatively coupled to the at least one processor and configured to detect a position of at least one of the patient, the care provider, or a visitor of the patient, wherein: the output device includes a screen mounted in a room associated with the patient, and the operations further include: causing the screen to visually output text including third information about the patient, a font size of the text being based on the position of at least one of the patient, the care provider, or the visitor.
13. The system of clause 12, further including: an input device configured to detect at least one of a voice or writing of at least one of the patient or a visitor of the patient, wherein the operations further include: identifying a language of the voice or the writing; and causing the output device to output third information about the patient in the language.
14. The system of any one of clauses 10 to 13, further including: an input device configured to detect a voice of the care provider; and a transceiver configured to transmit, to one or more electronic medical record (EMR) servers, EMR data associated with the patient and based on the voice of the care provider.
15. The system of clause 14, wherein the input device is configured to detect the voice as the care provider is treating the patient.
16. The system of any one of clauses 10 to 15, further including: a transceiver configured to receive, from one or more electronic medical record (EMR) servers, EMR data associated with the patient; and one or more sensors configured to detect a vital sign of the patient, wherein identifying the condition is based on at least one of the EMR data or the vital sign.
17. The system of any one of clauses 10 to 16, wherein: the condition includes at least one of a cardiac arrest or a respiratory arrest of the patient, and the second information includes a timer indicating an elapsed time since the condition was identified.
18. An electronic whiteboard, including: a screen physically mounted in a room associated with a patient; at least one processor communicatively coupled to the screen; at least one sensor communicatively coupled to the at least one processor and configured to detect a position of a first care provider and a position of a second care provider; an input device communicatively coupled to the at least one processor and configured to receive an input signal from the first care provider; a transceiver configured to transmit, to one or more electronic medical record (EMR) servers, data based on the input signal; memory communicatively coupled to the at least one processor and storing instructions that, when executed by the at least one processor, cause the at least one processor to perform operations including: determining that the position of the first care provider is at least one of within a threshold distance of the screen or within the room associated with the patient; causing the screen to output a first graphical user interface (GUI) associated with the first care provider; modifying the first GUI based on the input signal from the first care provider; determining that the position of the second care provider is at least one of within the threshold distance of the screen or within the room associated with the patient; based on determining that the position of the second care provider is at least one of within the threshold distance of the screen or within the room associated with the patient, causing the screen to output a second GUI associated with the second care provider, the second GUI being different than the first GUI.
19. The electronic whiteboard of clause 18, wherein the at least one sensor includes at least one of a microphone array configured to detect voices of the first care provider and the second care provider, a real time location system (RTLS) sensor configured to detect badges worn by the first care provider and the second care provider, or a camera configured to detect images of the first care provider and the second care provider.
20. The electronic whiteboard of clause 18 or 19, the data being first data, wherein: the transceiver is further configured to periodically receive, from the one or more EMR servers, second data indicating a condition of the patient, and at least one of the first GUI or the second GUI indicates the condition of the patient.
21. The electronic whiteboard of any one of clauses 18 to 20, wherein: the first GUI indicates first information and second information about the patient, and the second GUI indicates the first information without indicating the second information.
22. The electronic whiteboard of any one of clauses 18 to 21, wherein the operations further include: determining that the position of the first care provider and the position of the second care provider are at least one of greater than the threshold distance from the patient or outside of the room of the patient; based on determining that the position of the first care provider and the position of the second care provider are at least one of greater than the threshold distance from the patient or outside of the room of the patient: causing the screen to output a third GUI; or causing the screen to enter a dormant state displaying an absence of information about the patient.
23. A method, including: outputting, by an output device, a first Graphical User Interface (GUI) associated with a patient; adjusting a font size of the first GUI based on a position of the patient; determining that the position of a care provider is within a threshold distance of the output device or within a room associated with the patient; based on determining that the position of a care provider is within the threshold distance of the output device or within the room associated with the patient, outputting, by the output device, the second GUI being different than the first GUI.
24. The method of clause 23, further including: identifying, based on at least one of an electronic medical record (EMR) of the patient or one or more vital signs of the patient, a condition of the patient; based on identifying the condition of the patient, outputting, by the output device, a third GUI associated with the condition of the patient.
25. The method of clause 24, wherein: the condition of the patient includes at least one of cardiac arrest or respiratory arrest, and the third GUI includes a timer indicating an elapsed time since the condition was detected.
26. The method of any one of clauses 23 to 25, further including: receiving, by an input device, an input signal from the patient or the care provider; and updating the EMR of the patient based on the input signal.
27. The method of clause 26, further including: identifying a language of the input signal, wherein: at least one of the first GUI or the second GUI includes text in the language, and updating the EMR of includes updating the EMR based on the language.
28. The method of clause 26 or 27, wherein receiving the input signal includes detecting a voice of the care provider as the care provider is performing a treatment on the patient.
29. The method of any one of clauses 23 to 28, wherein the first GUI includes at least one of an identity of the care provider, contact information associated with the care provider, an ambulation instruction, educational materials about a condition of the patient, a care schedule of the patient, or a game related to the condition of the patient.
30. The method of any one of clauses clause 23 to 29, wherein the second GUI includes at least one of contact information of the patient, one or more care goals of the patient, a pain scale of the patient, one or more timers, diagnostic information about the patient, one or more tasks to be completed by the care provider, one or more conditions of the patient, one or more vital signs of the patient, or a discharge plan of the patient.

### CONCLUSION

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

What is claimed is:

1. A system, comprising:
an output device associated with a patient or a care provider;
at least one processor communicatively coupled to the output device;
memory communicatively coupled to the at least one processor and storing instructions that, when executed by the at least one processor, cause the at least one processor to perform operations comprising:
causing the output device to output first information about the patient;
identifying a condition of the patient; and
based on identifying the condition of the patient, causing the output device to output second information about the patient, the second information being different than the first information and comprising at least one of a timer or an instruction for treating the condition of the patient.

2. The system of claim 1, further comprising:
at least one sensor communicatively coupled to the at least one processor and configured to detect a position of the output device,
wherein:
the output device comprises an augmented reality (AR) device worn by the care provider and configured to output the first information and the second information in at least one AR overlay; and
the operations further comprise:
determining that the position of the output device is at least one of within a threshold distance of the patient or within a room associated with the patient; and
based on determining that the position of the output device is at least one of within the threshold distance of the patient or the room associated with the patient, causing the output device to output the at least one AR overlay.

3. The system of claim 1, further comprising:
at least one sensor communicatively coupled to the at least one processor and configured to detect a position of at least one of the patient, the care provider, or a visitor of the patient,
wherein:
the output device comprises a display mounted in a room associated with the patient, and
the operations further comprise:
causing the display to visually output text comprising third information about the patient, a font size of the text being based on the position of at least one of the patient, the care provider, or the visitor.

4. The system of claim 3, further comprising:
an input device configured to detect at least one of a voice or writing of at least one of the patient or a visitor of the patient,
wherein the operations further comprise:
identifying a language of the voice or the writing; and
causing the output device to output third information about the patient in the language.

5. The system of claim 1, further comprising:
an input device configured to detect a voice of the care provider; and
a transceiver configured to transmit, to one or more electronic medical record (EMR) servers, EMR data associated with the patient and based on the voice of the care provider,
wherein the input device is configured to detect the voice as the care provider is treating the patient.

6. The system of claim 1, further comprising:
a transceiver configured to receive, from one or more electronic medical record (EMR) servers, EMR data associated with the patient; and
one or more sensors configured to detect at least one of a vital sign of the patient, a fluid administered to the patient, or a medication administered to the patient,
wherein identifying the condition is based on at least one of the EMR data, the vital sign, the fluid, or the medication.

7. The system of claim 1, wherein:
the condition comprises at least one of a cardiac arrest or a respiratory arrest of the patient, and
the second information comprises a timer indicating an elapsed time since the condition was identified.

8. An electronic whiteboard, comprising:
a screen physically mounted in a room associated with a patient;
at least one processor communicatively coupled to the screen;
at least one sensor communicatively coupled to the at least one processor and configured to detect a position of a first care provider and a position of a second care provider;
an input device communicatively coupled to the at least one processor and configured to receive an input signal from the first care provider;
a transceiver configured to transmit, to one or more electronic medical record (EMR) servers, data based on the input signal; and
memory communicatively coupled to the at least one processor and storing instructions that, when executed by the at least one processor, cause the at least one processor to perform operations comprising:
determining that the position of the first care provider is at least one of within a threshold distance of the screen or within the room associated with the patient;
causing the screen to output a first graphical user interface (GUI) associated with the first care provider;
modifying the first GUI based on the input signal from the first care provider;
determining that the position of the second care provider is at least one of within the threshold distance of the screen or within the room associated with the patient; and
based on determining that the position of the second care provider is at least one of within the threshold distance of the screen or within the room associated with the patient, causing the screen to output a second GUI associated with the second care provider, the second GUI being different than the first GUI.

9. The electronic whiteboard of claim 8, wherein the at least one sensor comprises at least one of a microphone array configured to detect voices of the first care provider and the second care provider, a real time location system (RTLS) sensor configured to detect badges worn by the first care provider and the second care provider, or a camera configured to detect images of the first care provider and the second care provider.

10. The electronic whiteboard of claim 8, the data being first data, wherein:
the transceiver is further configured to periodically receive, from the one or more EMR servers, second data indicating a condition of the patient, and
at least one of the first GUI or the second GUI indicates the condition of the patient.

11. The electronic whiteboard of claim 8, wherein:
the first GUI indicates first information and second information about the patient, and
the second GUI indicates the first information without indicating the second information.

12. The electronic whiteboard of claim 8, wherein the operations further comprise:
determining that the position of the first care provider and the position of the second care provider are at least one of greater than the threshold distance from the patient or outside of the room of the patient; and
based on determining that the position of the first care provider and the position of the second care provider are at least one of greater than the threshold distance from the patient or outside of the room of the patient:
causing the screen to output a third GUI; or
causing the screen to enter a dormant state displaying an absence of information about the patient.

13. A method, comprising:
outputting, by an output device, a first Graphical User Interface (GUI) associated with a patient;
adjusting a font size of the first GUI based on a position of the patient;
determining that the position of a care provider is within a threshold distance of the output device or within a room associated with the patient; and
based on determining that the position of a care provider is within the threshold distance of the output device or within the room associated with the patient, outputting, by the output device, the second GUI being different than the first GUI.

14. The method of claim 13, further comprising:
identifying, based on at least one of an electronic medical record (EMR) of the patient or one or more vital signs of the patient, a condition of the patient; and
based on identifying the condition of the patient, outputting, by the output device, a third GUI associated with the condition of the patient.

15. The method of claim 14, wherein:
the condition of the patient comprises at least one of cardiac arrest or respiratory arrest, and
the third GUI comprises a timer indicating an elapsed time since the condition was detected.

16. The method of claim 13, further comprising:
receiving, by an input device, an input signal from the patient or the care provider; and
updating the EMR of the patient based on the input signal.

17. The method of claim 16, further comprising:
identifying a language of the input signal,
wherein:
at least one of the first GUI or the second GUI comprises text in the language, and
updating the EMR of comprises updating the EMR based on the language.

18. The method of claim 16, wherein receiving the input signal comprises detecting a voice of the care provider as the care provider is performing a treatment on the patient.

19. The method of claim 13, wherein the first GUI comprises at least one of an identity of the care provider, contact information associated with the care provider, an ambulation instruction, educational materials about a condition of the patient, a care schedule of the patient, or a game related to the condition of the patient.

20. The method of claim 13, wherein the second GUI comprises at least one of contact information of the patient, one or more care goals of the patient, a pain scale of the patient, one or more timers, diagnostic information about the patient, one or more tasks to be completed by the care provider, one or more conditions of the patient, one or more vital signs of the patient, or a discharge plan of the patient.
